(19) United States
(12) Patent Application Publication
(10) Pub. No.: US 2016/0310231 A1
(43) Pub. Date: Oct. 27, 2016

(54) Medical Magic Marker

(71) Applicant: Lee Cohen, San Marcos, CA (US)

(72) Inventor: Lee Cohen, San Marcos, CA (US)

(21) Appl. No.: 13/464,111

(22) Filed: May 4, 2012

(51) Int. Cl. A61B 90/80; A61L 31/16; A01N 33/12; A61L 2/00; A01N 65/08; A61L 31/00; C09D 11/17

(52) U.S. Cl. CPC A61B 90/80; A61L 31/005; A61L 31/16; C09D 11/17; A61L 2/0088; A01N 65/08; A01N 33/12; A61L 2300/404; A61L 2300/30; A61L 2300/208; A61L 2300/442; A61L 2300/802

(57) ABSTRACT

The present invention relates to an apparatus and method for dispensing at least one fluid for use in reducing the bacteria count on hands during washing and providing an objective measure of cleanliness of hands. The preferred embodiment of the apparatus being in a shape of a magic marker pen comprising at least one chamber which contains a non toxic sterile ink like material that, when applied to a person's hands disappears after a certain amount of time washing and a felt-tip for dispensing said material. The non toxic sterile ink contained in the preferred embodiment is a natural product of Quercus Infectoria or similar which is also an antimicrobial reagent against Staphylococcus, Pseudomonas and common viral agents. In use, after placing a mark on both palms of his/her hands using the apparatus, the user would wash/wipe hands as described above until the ink disappears.

### BACKGROUND OF THE INVENTION

1. Field of the Invention

The present invention relates to an apparatus and method for dispensing at least one fluid for use in reducing the bacteria count on hands during washing where a non toxic sterile ink like material that, when applied to a person's hands disappears after a certain amount of time washing and can include an antimicrobial fortifier for the common organisms of Staphylococcus, Pseudomonas and common viral agents. The sterile ink like material will come from a well-known and studied safe tannin plant material known as Quercus Infectoria and or similar in which the ink/dye like material will dissolve over a specified time as the hands are being washed and result in both a methodology for ensuring proper hand washing and a fortifying agent such as Quercus Infectoria and or similar which has numerous publications on its safety and its effectiveness against many of the nosocomial transmittable agents such as Staphylococcus and Methicillin resistant Staphylococcus bacterium.

2. Description of Related Art

The transmission of microorganisms from the hands of health care workers is the main source of cross infection in hospitals and health care facilities and can be prevented by hand washing. The recent awareness and endemic spread of H1N1 and other similar infections of public awareness has created a tremendous demand for proper cleansing that is both practical and effective against these offenders above as well as others.

In the United States, approximately 46.5 million surgical procedures and even more invasive medical procedure are performed each year. Each procedure involves contact by medical personnel with a patient's sterile tissue or mucous membranes. A major risk of all such procedures is the introduction of pathogens that can lead to infection. Failure to properly hand wash carries not only risk associated with breach of host barriers but also risk for person-to-person transmission (e.g., hepatitis B virus) and transmission of environmental pathogens (e.g., Pseudomonas Aeruginosa).

Hand washing is emphasized as the single most important measure to prevent cross transmission of micro-organisms and thus to prevent nosocomial infections. However, under routine hospital practice compliance with this measure is still unacceptably low, less than 50% in most studies published in the past 20 years. This constant finding is worrying because recent studies have shown that this level of compliance will not reduce the risk of transmission of multi-resistant bacteria in hospital. Attempts to improve compliance have included increasing the number of accessible sinks, and educating healthcare workers, but none of these interventions led to a marked and sustained improvement in compliance.

Surgeons have witnessed the results of infections due to poor hygiene administered by many medical personnel and have sat on infectious control committees pleading with the staff for proper hand cleansing and sterilization.

Both in medical School and in surgical training doctors are taught to scrub their hands a certain way for a certain amount of time. Nevertheless, there is no objective measure of cleanliness or accountability. Surgeons simply relied on vigorous washings. That is the current standard today; there is no objective measure or indicator of cleanliness or accountability.

Unfortunately statistics and studies show that improper hand cleansing is responsible for a majority of hospital infections leading to increased deaths and tremendous costs to the health care system. It may include thousands of lives and over one billion dollars in unnecessary costs due to improper hygiene and the spread of these infections.

Accordingly, there is a need for a method and apparatus that can reduce the microbes on the hands and provide an objective measure or indicator of cleanliness or accountability, thus helping to save lives and unnecessary costs due to improper hygiene and the spread of the infections.

### SUMMARY OF THE INVENTION

The object of the present invention is to provide a method and a specific apparatus to reduce the microbes on the hands and determine whether medical staff's hands are surgically sterile ultimately avoiding the transmission of infections. Another object of the present invention is to provide a method and a specific apparatus to reduce the microbes on consumers' hands avoiding the transmission of infections.

In an exemplary embodiment of the present invention deemed as medical/surgical healthcare version, there is disclosed apparatus for dispensing at least one fluid for use in reducing the bacteria count on hands and providing objective measure of cleanliness during washing comprising:

At least one chamber having a non toxic sterile ink like material (Quercus and or similar) that, when applied to a person's hands fortifies the active ingredient (e.g. Chlorohexidine and/or Povidone Iodine) which are already standardized in the industry. Chlorohexidine and or Povidone Iodine will be separately supplied via sinks and other areas for all medical and surgical personnel to use as they have in the past with no changes for hand washing techniques except for Quercus Ink/dye marker to be placed on the skin as an X and through time release be removed by hand washing with accepted health care hand washing in the facility to ensure proper hand washing time and accountability as well as a fortifying agent such as Quercus and or similar to enhance and fortify against common nosocomial bacteria transmission of Staphylococcus and methicillin resistant Staphylococcus in the health care setting for the common bacterial and viral agents (see attached hereto papers in support); and
Means for dispensing said material coupled to an end of said chamber.

In another exemplary embodiment of the present invention deemed as the Consumer variation to be used in all consumer applications for proper hand washing there is disclosed a method for dispensing at least one fluid for use in reducing the bacteria count on hands and providing objective measure of cleanliness during washing comprising:
providing at least one chamber or a mixed chamber having a non-toxic sterile ink like material (Again the Quercus Infectoria or similar) that, when applied to a person's hands disappears after a certain amount of time washing and an antimicrobial reagent for the common bacterial and viral agents that has been standardized and accepted by the FDA and other governing authorities that is natural and non-alcohol based

The FDA accepted the use of both alcohol and Benzalkonium Chloride (BAC) as an active germ killing ingredient for first aid antiseptics The FDA accepts the use of Benzalkonium Chloride (BAC)) in concentrations of 0.1 to 0.13 percent as an active germ killing ingredient for hand washing here in the USA

The active ingredient and concentration of (BAC) or other accepted non-alcohol based solution fortified with published tannin such as Quercus Infectoria known for its antibacterial properties against staphylococcus and methicillin resistant staph used as both a fortifier and as used in to make the physical X on our Medical magic marker makes this product unique.
and providing means for dispensing said material coupled to an end of said chamber.

The non toxic sterile ink is commercially available at different concentrations. The sterile ink, in one embodiment can be a reagent such as a homeopathic plant product extracted from Quercus Infectoria or similar natural antimicrobial tannin/dye. Galls of Quercus Infectoria, Olivier are used for dyeing. They also demonstrate a broad spectrum of activity against bacteria including Staphylococcus Aureus, Pseudomonas Aeruginosa, E. Coli, and Streptococcus, etc. Tannins are also known to have broad spectrum of antibacterial and antiviral activities. Other homeopathic plant extract products if appropriate may be used for the intended purpose.

The preferred embodiment of the apparatus, Magical Medical Marker, being in a shape of a magic marker has a reservoir (chamber) with a felt tip at the end and a snap on cover over the felt tip which can be clipped in place in a user's pocket. The reservoir (chamber) contains the non-toxic sterile ink/Quercus Infectoria and may contain other materials including antimicrobial reagent, soap, emollient, vitamin, Aloe, etc. The non-toxic sterile ink in the preferred embodiment is extract from Quercus Infectoria which also has antibacterial and antiviral activities.

To use, a user such as a Surgeon/Health care worker simply puts a mark such as an X, an X in a circle, a circle, a Z or any other mark on both palms of the user's hands with the marker and washes his/her hands in the usual manner until the mark such as an X disappears due to the time factor of the Ink to wear off as well as the Staphylococcus bacteria population goes to near zero levels as the hands are washed and the active accepted ingredients of Chlorohexidine, Povidone Iodine or other accepted and provided health care facility cleanser go to work. The X which is placed on by the Surgeon/Healthcare worker will be used as fortifier against Staphylococcus and methicillin resistant Staphylococcus and will also work as timed device to ensure proper hand washing. This means that as the X wears off, a methodology of proper hand wash timing using the accepted and already standardized solutions in health care facilities (IE chlorohexidine/Iodine) and Quercus as an antimicrobial fortifier is achieved.

A timed concentration to be effective against Staphylococcus and other bacteria and a timed rate of dissolution of the X on the hands to ensure proper hand wash time. The key issue in ineffective hand washing in health care facilities is both poor hand washing techniques coupled with rushing and not letting the hands wash with friction and time with the appropriate accepted hand washing solutions provided with the soaps and available solutions in the health care facility. This apparatus and method of the present invention ensures clean and sterile hands, and can be uniformly used in all healthcare and industrial applications such as, for example, operating suites, hospital rooms, kitchens, intensive care areas, and all outpatient and inpatient health care facilities. In another application, the disclosed invention can be used in consumer and industrial applications such as computer chip manufacturing, food plants, schools, and/or any other public or private gathering.

Use of the Magical Medical Marker will set the standard for once on what is acceptable, will change the world by saving hundreds of thousands of unnecessary infections and deaths, and cut health care costs astronomically so our focus can be on healthy living.

The FDA has accepted the use of both alcohol and Benzalkonium Chloride (BAC) as an active germ killing ingredient for first aid antiseptics The FDA accepts the use of Benzalkonium Chloride (BAC)) in concentrations of 0.1 to 0.13 percent as an active germ killing ingredient for hand washing here in the USA.

The active ingredient and concentration of (BAC) or other accepted non-alcohol based solution fortified with published tannin such as Quercus Infectoria known for its antibacterial properties against staphylococcus and methicillin resistant staph used as both a fortifier and as used in to make the physical X on our Medical magic marker makes this product unique

Consumer Variant

The active ingredient is BAC and the fortifier is Quercus Infectoria and or similar. The Quercus being Tannin also is the Marker aspect of the device replacing the need and use of Sterile Ink. Note cited papers on its safety hereto attached. This is ecofriendly, green, natural and will also include other well know natural ingredients such as Aloe Vera Vit E and or similar. There will be only one chamber and the solution will be premixed in marker for simplicity and consistency.

Medical Variant

This variant will not need the BAC or any other solution other than the Quercus and or similar tannin and is to be used both as an indicator (marking device) like a sterile ink and a fortifier to ensure against staph and or similar nosocomial bacteria and or viruses known in this field as the soap portion will be what is acceptable as the standard of care such as chlorohexidine, povidone Iodine etc. . . . that is administered and hand wash as usual.

The medical device is simply the Quercus as an indicator and marker timed on the hand to ensure hand washing time and compliance with the added benefit of Quercus as a Fortifier not the active ingredient.

This again will be disposable and non-toxic.

The proprietor marker device has already the BAC as the active ingredient in its design for meeting regulations of hand washing according to regulatory bodies including the FDA CDC and World health Organization.

The addition of the Quercus Infectoria and or similar tannin which has numerous publications of its safety and efficacy against many of the common Pathogens in nosocomial infections including Staphylococcus Aureus and Methicillin resistant Staphylococcus makes the elimination of bacteria on the hands more substantial.

The Quercus and or similar is used as the X marking device as well and is applied in both the medical setting and the consumer setting clearly on different concentrations as to wipe off sooner and near immediately on the consumer version, and like a sterile ink in higher concentrations can be made to last up to 15 minutes if needed in surgical hand wash or any amount of time in-between.

The product ingredients will be eco-friendly, green, nontoxic, disposable with leaving minimal carbon foot print in its design and manufacturing.

The Medical Magic Marker is disposable, cost effective, non-toxic, environmentally friendly, portable, and can fit in any shirt pocket for travel.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is an overall view of a marker pan like instrument.

FIG. 2 is an overall view of a snap on cover having a clip.

### DESCRIPTION OF THE PREFERRED EMBODIMENT

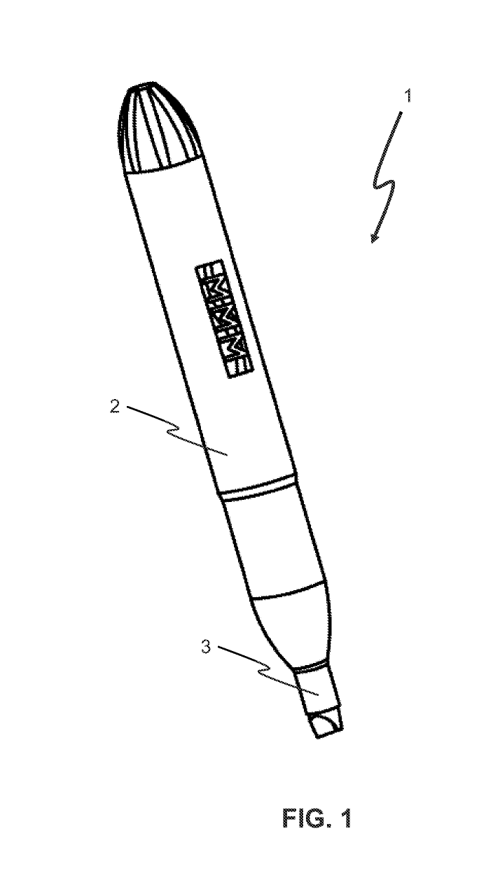

In an exemplary embodiment of the present invention, FIG. 1, there is disclosed apparatus 1 for dispensing at least one fluid for use in reducing the bacteria count on hands during washing comprising:
at least one chamber 2 having a non toxic sterile ink like material that, when applied to a person's hands disappears after a certain amount of time washing; and
means for dispensing said material 3 coupled to an end of said chamber.

The apparatus may further comprise antimicrobial reagents for the common microorganisms in said chamber or a separate chamber. The apparatus may further comprise a soap solution in said chamber or a separate chamber.

In another exemplary embodiment of the present invention, there is disclosed a method for dispensing at least one fluid for use in reducing the bacteria count on hands during washing comprising:
Providing at least one chamber having a non toxic sterile ink like material that when applied to a person's hands disappears after a certain amount of time washing; and
Locating means for dispensing said material coupled to an end of said chamber.

Wherein said at least one chamber may further include antimicrobial reagents for the common microorganisms and/or a soap solution within the same chamber or separate chambers.

A mechanism for delivering a fluid to dispense on a user's hands is not discussed here as this is known to those skilled in the art.

Before a person scrubs or washes his/her hands, he/she simply puts a mark such as an X, an X in a circle, a circle, a Z or any other mark on both palms of the user's hands with the apparatus. The apparatus contains a non toxic sterile ink like material that simply disappears over a certain amount of time washing and has a antimicrobial reagent for the common organisms of Staphylococcus, Pseudomonas and common viral agents i.e. hepatitis, H1N1 virus, and HIV. When these colonies of bacteria and viruses are reduced to near zero counts the reagent disappears. Therefore when the X on the palms disappears, so does the colonization of these microorganisms, and a true objective method of proper hand washing is established.

In an embodiment, the marker contains a non toxic homeopathic plant product called Quercus Infectoria or similar that simply disappears over a certain amount of time washing and is antimicrobial for the common organisms of Staphylococcus, Methicillin resistant Staphylococcus and Pseudomonas.

A consumer variant Magical Medical Marker can have 1, 2 or 3 reservoirs of chambers or any combination of chambers where one chamber may have a sterile timed ink (Quercus Infectoria and or similar) the second chamber may have the Aloe Vera and or Vitamin E emollients or similar and the third chamber may contain a soap/non-alcohol mix to be used when no running water is available i.e.; on a train, a bus, out in public in remote areas in developed world, in third world countries without running water. This chambers active ingredient will be The FDA accepted the use of both alcohol and Benzalkonium Chloride (BAC) as an active germ killing ingredient for first aid antiseptics. The FDA accepts the use of Benzalkonium Chloride (BAC)) in concentrations of 0.1 to 0.13 percent as an active germ killing ingredient for hand washing here in the USA

The active ingredient and concentration of (BAC) or other accepted non-alcohol based solution fortified with published tannin such as Quercus Infectoria known for its antibacterial properties against staphylococcus and methicillin resistant staph used as both a fortifier and as used in to make the physical X on our Medical magic marker makes this product unique

It is to be noted that all the ingredients above may well be mixed into one chamber as a stable solution, and the use of multiple chambers be unnecessary.

### The Medical/Surgical Healthcare

Apparatus would be as above but with only 1 chamber which may include a longer timed sterile ink/dye Quercus Infectoria and or similar in the chamber

The sterile ink/dye in one embodiment can be ingredients from Quercus Infectoria or similar. The Quercus Infectoria is also antimicrobial for the common microorganisms, such as Staphylococcus, Pseudomonas and common viral agents i.e. hepatitis, H1N1 virus, HIV, etc.

The apparatus can be refilled and reused or disposed when the ingredients run out.

This variant will not need the BAC or any other solution other than the Quercus and or similar tannin and is to be used as the marking device (instead of the sterile ink) and to be used as both a fortifier to ensure against staph and or similar nosocomial bacteria and or viruses know in this field as the soap portion will be what is acceptable as the standard of care such as chlorohexidine, povidone Iodine etc. . . . that is administered and hand wash as usual.

The medical device is simply the Quercus as an indicator and marker timed on the hand to ensure hand washing time and compliance with the added benefit of Quercus as a Fortifier not the active ingredient.

This again will be disposable and non-toxic.

The proprietor marker device has already the BAC as the active ingredient in its design for meeting regulations of hand washing according to regulatory bodies including the FDA CDC and World health Organization.

The addition of the Quercus Infectoria and or similar tannin which has numerous publications of its safety and efficacy against many of the common Pathogens in nosocomial infections including Staphylococcus Aureus and Methicillin resistant Staphylococcus makes the elimination of bacteria on the hands more substantial.

The Quercus and or similar is used as the X marking device as well and is applied in both the medical setting and the consumer setting clearly on different concentrations as to wipe off sooner and near immediately on the consumer version, and like a sterile ink in higher concentrations can be made to last up to 15 minutes if needed in surgical hand wash or any amount of time in-between.

The product ingredients will be eco-friendly, green, nontoxic, disposable with leaving minimal carbon foot print in its design and manufacturing.

All proprietary ingredients included in the chamber(s) are natural proven non-toxic green elements that are bio-degradable.

Different concentrations would be used in different settings and take different washing time to wear off. For example, surgical scrub for surgeon would last about 12-15 minutes; Nurses washing hands before handling patients about 5 minutes; and consumer use may be instant.

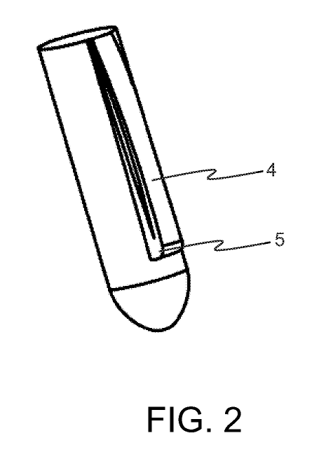

In the preferred embodiment, the Medical magic marker can look like a magic marker, a marker pen like instrument (a medical pen version 1) having a reservoir (chamber) 2 with a felt tip 3 at the end as shown in FIG. 1. The medical pen version 1 further comprises a snap on cover 4 (as shown in FIG. 2) to be placed over the felt tip. The medical pen version 1 further has a clip 5 (as shown in FIG. 2) attached to the snap on cover 4 such that it can be clipped in place in a user's pocket and or be attached to a key chain like a small marking device which is more portable and easy to carry. In the case that when the Magical Medical Marker has only one chamber, all ingredients including a sterile ink, and/or antimicrobial reagents, and/or a soap may be held in the same chamber.

A mechanism for delivering a fluid to the felt tip and dispensing on a user's hands is not discussed here as this is within the knowledge of one familiar with a magical marker.

The Magical Medical Marker version (professional healthcare variant) can have Quercus Infectoria or similar natural antimicrobial tannin/dye along with Aloe Vera and Vitamin E. In one embodiment, after the marker is applied, scrubbed, washed, the color disappears (dissolves) in 5-15 minutes.

The Magical Medical Marker version (consumer variant) will have less concentration of Quercus Infectoria or similar natural antimicrobial tannin/dye, Aloe Vera, Vitamin E but will also include a natural soap or emollient as to not require hand washing with outside water and will be smaller than the medical variant and fit on a key chain ring for portability and will work the same way with a wick embedded with these ingredients and essentially wiping hands eliminated the X almost immediately.

The only near competitor is the bulky soap and alcohol dispensers such as Purell, sitting on public facility counters and bathrooms. However, the exterior surfaces of these bulky soap and alcohol are full of germs by way of massive public hand use, and also do not contain any indicators whether one's hands are truly germ free and can include alcohol with drying effects on the hands. There may be hand sanitizers available in the marketplace, but they do not include any indicators (color monitoring system) to show whether one's hands are truly germ free.

In use, after placing a mark such as an X on both palms of his/her hands, the user would wash or wipe his/her hands until the X is no longer present

In another embodiment and in addition in the health care arena, after the X gets washed off an extra assuring step can be with the user placing his/her hands under a low level ultraviolet light for short period of time. UV light is known to be toxic to these bacteria, and whatever small amounts remain will be killed. This is an optional step.

This apparatus and method disclosed above would be the new standard of proving that hands are free of bacteria and would assure compliance and patient safety.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are entitled.

What is claimed is:

1. Apparatus for dispensing at least one fluid for use in reducing the bacteria count on hands and providing objective measure of cleanliness during washing comprises:
at least one chamber having a non toxic sterile inks like material that, when applied to a person's hands disappears after a certain amount of time washing; and
means for dispensing said material coupled to an end of said chamber.

2. The apparatus of claim 1, wherein the at least one chamber further includes antimicrobial fortifiers against common microorganisms.

3. The method of claim 1 wherein the non toxic sterile inks include homeopathic plant extracts products that are both antimicrobial fortifiers and dyes.

4. The apparatus of claim 1, wherein the non toxic sterile inks like material include homeopathic plant products extracted from Quercus Infectoria and/or similar natural antimicrobial tannins/dyes.

5. The apparatus of claim 2, wherein the antimicrobial fortifiers include homeopathic plant products extracted from Quercus Infectoria and/or similar natural antimicrobial tannins/dyes.

6. The apparatus of claim 1, wherein the at least one chamber further includes a soap solution which includes but is not limited to Benzalkonium Chloride in the consumer variation.

7. The apparatus of claim 6, wherein the at least one chamber further includes natural emollient, Vitamin E, and Aloe Vera.

8. The apparatus of claim 1, wherein the at least one chamber can open individually and separately at an end, or the fluid in each chamber can be channeled to a single chamber where they are combined and dispensed from a single opening or any combination set forth with these parameters.

9. The apparatus of claim 1 having a shape of a marker pen with a felt tip at the end, a cover over the felt tip, and a snap on the cover which can be clipped in place in a user's pocket and/or be attached to a key chain like a small marking device.

10. A method for dispensing at least one fluid for use in reducing the bacteria count on hands and providing objective measure of cleanliness during washing comprises:
providing at least one chamber having non toxic sterile inks like material that, when applied to a person's hands disappears after a certain amount of time washing; and
providing means for dispensing said material coupled to an end of said chamber.

11. The method of claim 10 further comprises providing antimicrobial fortifiers against common microorganisms in the at least one chamber.

12. The method of claim 10 wherein the non toxic sterile inks include homeopathic plant products extracted from Quercus Infectoria or similar natural antimicrobial tannins/dyes.

13. The method of claim 10 wherein the antimicrobial fortifiers include homeopathic plant products extracted from Quercus Infectoria or similar natural antimicrobial tannins/dyes.

14. The method of claim 10 further comprises providing an antiseptic soap solution including but not limited to Benzalkonium Chloride in the at least one chamber.

15. The method of claim 14 further comprises providing natural emollient, Vitamin E, and Aloe Vera in the at least one chamber.

16. The method of claim 10, wherein the at least one chamber can open individually and separately at an end, or the fluid in each chamber can be channeled to a single chamber where they are combined and dispensed from a single opening or any combination set forth with these parameters.

17. The method of claim 10 further comprising providing a shell to enclose the at least one chamber, a felt tip coupled at the end of the shell, a cover over the felt tip, and a snap on the cover which can be clipped in place in a user's pocket and/or be attached to a key chain like a small marking device.

18. The apparatus of claim 1, wherein the sterile ink/and/or Quercus Infectoria has different concentrations depending on the settings.

19. The apparatus of claim 18, wherein the concentration of the sterile ink and/or Quercus Infectoria being made to time an effective hand washing based on the concentration and dissolution rate of the sterile ink and/or Quercus Infectoria.

20. The apparatus of claim 19, wherein the concentration of sterile ink and/or Quercus Infectoria for medical staff can be made up to about 15 minutes in surgical hand wash or any amount of time in-between.
